SEQUENCE LISTING (3 sequences)

SEQ ID NO: 1 - DNA, 20 bp, Homo sapiens
gggacgcggcgctcggtcat

SEQ ID NO: 2 - RNA, 22 nt, Homo sapiens
aagucucaucggauuuugcagc

SEQ ID NO: 3 - RNA, 21 nt, Artificial Sequence
  scrambled control siRNA
cagcgcugacaacaguuucau

(19) United States
(12) Patent Application Publication
(10) Pub. No.: US 2009/0281166 A1
(43) Pub. Date: Nov. 12, 2009

(54) Treatment of Cancer by Inhibition of HSP27

(75) Inventors: Martin Gleave, Vancouver (CA); Christopher Ong, Vancouver (CA); Norihiro Hayashi, Vancouver (CA)

Correspondence Address: Larson & Anderson, LLC, P.O. BOX 4928, DILLON, CO, 80435, US

(73) Assignee: THE UNIVERSITY OF BRITISH COLUMBIA, Vancouver (CA)

(21) Appl. No.: 12/423,359

(22) Filed: Apr. 14, 2009

Related U.S. Application Data
(60) Provisional application No. 61/045,269, filed on Apr. 15, 2008.
(60) Provisional application No. 61/044,868, filed on Apr. 14, 2008.

(51) Int. Cl. A61K 31/7088; C12Q 1/68; C12Q 1/42; A61K 31/519; A61K 31/435; A61K 31/4355; A61K 31/05

(52) U.S. Cl. 514/44 A; 435/6; 435/21; 514/44 R; 514/257; 514/279; 514/280; 514/733

(57) ABSTRACT

A cancer is evaluated for selection of appropriate therapy by evaluating a sample of cancerous tissue to determine an expression of level of phosphatase and tensin homologue deleted from chromosome 10 (PTEN); and in the case where the expression level of functional PTEN is below a threshold level, identifying the cancer as susceptible to an active agent that inhibits the expression of heat shock protein 27 (hsp27). The evaluation may be included as part of a method of treatment, in which an hsp27 inhibitor is selected as a therapeutic agent when the PTEN level is below the threshold.

This application claims the benefit of U.S. Provisional Applications Nos. 61/044,868 filed Apr. 14, 2008, and 61/045,269 filed Apr. 15, 2008, both of which are incorporated herein by reference in their entirety.

### BACKGROUND OF THE INVENTION

This application relates to the treatment of cancer through the inhibition of heat shock protein 27 (hsp27).

Hsp27 is a cell survival protein found at elevated levels in many human cancers including prostate, lung, breast, ovarian, bladder, renal, pancreatic, multiple myeloma and liver cancer. In addition, many anti-cancer therapies are known to further elevate Hsp27 levels. For example, Hsp27 levels increased four-fold in prostate cancer patients after treatment with chemo- or hormone therapy. Increased levels of Hsp27 in some human cancers are associated with metastases, poor prognosis and resistance to radiation or chemotherapy.

Hsp27 has been disclosed as a therapeutic target in the treatment of cancer. For example, U.S. Pat. No. 7,101,991 discloses antisense oligonucleotides and siRNA that inhibit hsp27 expression. Additional oligonucleotide sequences targeting hsp27 expression are disclosed in WO2007/025229. Non-oligonucleotide compounds for inhibition of hsp27 have also been disclosed, including berberine derivatives described in European Patent EP0813872, and compounds described in JP 10045572, JP 10045574, JP10036261 and JP 10036267, all assigned to Kureha Chemical Industries Co,. Ltd. Paclitaxel has also been shown to be an inhibitor of hsp27 expression. Tanaka et al., Int J Gynecol Cancer. 2004 Jul-Aug; 14(4): 616-20.

Preclinical studies show that OGX-427, an antisense oligonucleotide described in U.S. Pat. No. 7,101,991 (Seq. ID No. 1, OncoGenex Technologies Inc.), significantly decreases levels of Hsp27, induces apoptosis in several human cancer cell lines, has single agent anti-tumor activity, and acts as a chemosensitizer in combination with several cytotoxic drugs including docetaxel. OGX-427 is being evaluated in a Phase 1 study in patients with breast, prostate, ovarian, non-small cell lung, or bladder cancer who have failed potentially curative treatments or for which a curative treatment does not exist.

### SUMMARY OF THE INVENTION

The present inventors have now found that the status of the tumor suppressor protein referred to as phosphatase and tensin homologue deleted from chromosome 10 (PTEN) in the target cancer cells effects the activity of the hsp27 as a therapeutic. Specifically, as demonstrated below, in PTEN deficient/negative cancer cell lines, hsp27 inhibition is observed, while no statistical benefit is observed from hsp27 inhibition when functional PTEN protein is present in the target cells. Accordingly, the present invention provides a method for the treatment of cancer using hsp27 inhibition that includes a preliminary test to ascertain the status of the PTEN in the target cells.

In accordance with the present invention there is provided a method for treating cancer in a patient diagnosed as suffering from cancer comprising the steps of:

(a) obtaining a sample of cancerous tissue from the patient;

(b) evaluating the sample of cancerous tissue to determine an expression of level of functional PTEN; and

(c) in the case where the expression level of functional PTEN is below a threshold level, administering to the patient a therapeutic composition comprising as an active agent a composition effective to inhibit the expression or activity of hsp27. The nature of the active agent is not critical, although in certain specific embodiments, the active agent is an antisense oligonucleotide (such as ONGX-427, Oncogenex) or a duplex siRNA.

The invention further provides for the use of a PTEN level assay and an HSP27 inhibitor for the treatment of cancer. The invention further provides for the use of PTEN expression levels in a tumor/cancerous tissue for the selection of treatment strategy using an HSP27 inhibitor.

### BRIEF DESCRIPTION OF THE DRAWINGS

FIGS. 1A and B show increased proliferation in LNCaPHsp27 cells, as compared to LNCaPMock cells as observed by [³H]-thymidine incorporation and cell counting, respectively.

FIGS. 2, 3A and B show % of cells in sub G1 after treatment with CH-11, Scr antisense, siHsp27 and CHX.

FIG. 4 shows differential cell counts for cells lines with and without PTEN after treatment with siHsp27.

FIGS. 5A and B shows effects of siHsp27 in a cell line with inducible PTEN.

### DETAILED DESCRIPTION OF THE INVENTION

The present invention provides a method of for treating cancer in a patient diagnosed as suffering from cancer. In preferred embodiments, the patient is a human patient, although the method can also be used in veterinary applications, for example in the treatment of cancer in dogs, cats and other pets.

As used in the specification and claims of the present application, the term “treating” refers to performing the method steps of the invention with intention and expectation of a therapeutic benefit to the patient. It would be understood in the art that not all patients respond favorably, or to the same extent to a given treatment. Furthermore, it will be understood in the art that the results of obtained for any individual can not be compared to results for that individual in the absence of the treatment. Thus, actual therapeutic benefit is not required to fall within the scope of the concept of “treating.”

The occurrence of elevated levels of hsp27 in various types of cancer and the demonstrated efficacy of hsp27 inhibitors in multiple types of cancers is indicative of the general applicability of the present invention to cancers of many types. In general, the method will be employed with cancer types which are considered to be targets for hsp27 therapy, including in particular those where there has been a previous determination of hsp27 overexpression for the patient's cancer. Specific non-limiting examples of cancer types that may be treated using the method of the invention include breast, prostate, ovarian, uterine, non-small cell lung, bladder, gastric, liver, endometrial, laryngeal and colorectal cancers; squamous cell carcinomas such as esophageal squamous cell carcinoma, glioma, glioblastoma, melanoma, multiple myelmoma and lymphoma.

The first step of the present method is obtaining a sample of cancerous tissue from the patient for evaluation. Such samples can be obtained using normal biopsy and sampling techniques consistent with the type of cancer. The size of the sample needed is based upon the evaluation procedure to be employed.

Once a sample of cancerous tissue is obtained it is evaluated to determine an expression of level of functional PTEN. As used herein, the term “functional PTEN” refers to PTEN that retains its wild-type ability to inhibit the phosphatidylinositol 3′-kinase/Akt pathway and hence to act as a tumor suppressor. Reduced levels of functional PTEN may result from decreases in the total amount of PTEN expressed, from modifications to expressed PTEN (for example methylation of PTEN as reported by Mirmohammadsadegh et al, Cancer Res. 2006: 66(1) 6546-52), or from mutations in the PTEN gene that result in the expressed protein being defective.

There are numerous methods by which the level of functional PTEN may be determined including immunohistochemical methods, polymerase chain reaction (PCR analysis), and PTEN specific immunoassays such as PTEN ELISA. Examples of specific known assays include without limitation those in the citations listed in Table 1, all of which are incorporated herein by reference.

TABLE 1

| Cell Type | Assay Type | Citation |
|---|---|---|
| Breast | | Cell Oncol. 2007; 29(1): 25-35. |
| Breast | immunohistochemical | Histopathology. 2006 September; 49(3): 248-55 |
| Breast | methylation-specific | Genes Chromosomes Cancer. 2004 |
| | PCR assay | October; 41(2): 117-24 |
| Breast | | Breast Cancer Res. 2001; 3(6): 356-60. |
| Breast | tissue microarray | Arch Pathol Lab Med. 2007; 131: 767-772 |
| Endometrial | immunocytochemical | Int J Gynecol Cancer. 2007 |
| | | May-June; 17(3): 697-704 |
| Endometrial | DNA sequencing | Clin Cancer Res. 2006 Oct 15; 12(20 Pt 1): 5932-5. |
| Esophageal | immunohistochemical | Dis Esophagus. 2007; 20(6): 491-6. |
| Squamous Cell | | |
| Carcinoma | | |
| Gastric | tissue microarray | Appl Immunohistochem Mol Morphol. 2007 |
| | | December; 15(4): 432-40. |
| Gastric | | Int J Cancer. 2008 Jan 15; 122(2): 433-43 |
| Gastric | immunohistochemical | World J Gastroenterol. 2006 Feb 21; 12(7): 1013-7 |
| Glioma | RT-PCR | Scand J Clin Lab Invest. 2006; 66(6): 469-75. |
| Glioma | SSCP and sequencing | Cancer Res. 1997 Oct. 1; 57: 4187-4190. |
| Glioblastoma | | Cancer Res. 2007 May 1; 67(9): 4467-73 |
| liver | | Liver Int. 2007 March; 27(2): 155-62 |
| Non-Small Cell | immunohistochemical | Oncol Rep. 2007 April; 17(4): 853-7 |
| Lung Cancer | | |
| Melanoma | RT-PCR | Cancer Res. 2006 Jul 1; 66(13): 6546-52 |
| breast, prostate, | immunohistochemical | Proc Natl Acad Sci USA. 2007 May |
| and bladder | | 1; 104(18): 7564-9. |
| carcinoma | | |
| renal cell | tissue microarray | Pathology. 2007 October; 39(5): 482-5 |
| carcinoma and | | |
| oncocytoma | | |
| astrocytoma | yeast-based assay for | Oncogene. 2000 Sep 7; 19(38): 4346-53 |
| | the detection of PTEN | |
| | nonsense mutation | |
| colorectal | | Br J Cancer. 2007 Oct 22; 97(8): 1139-45 |

ELISA test kits for PTEN are commercially available: PTEN ELISA Assay Kit from Echelon Biosciences Inc., Salt Lake City, Utah and Human/Mouse/Rat PTEN ELISA development Kit, DuoSet, IC (intracellular), Minneapolis, Minn. (Catalog Number DYC847-2). Materials for immunohistochemical assays for PTEN are also available commercially: Pathway Diagnostics, a cell staining assay Phosphatase and Tensin Homolog (PTEN); PTEN (clone 17A, NeoMarkers; ready-to-use) and SP kit from Fujian Maxin Ltd (China); and monoclonal PTEN antibody, 6H2.1, from Cascade BioScience, Inc, Winchester, Mass. Other PTEN monoclonal antibodies are available from Neomarkers and Zymed. See Modern Pathology 2005; 18: 719-727 which is incorporated herein by reference. An RT-PCR kit for PTEN detection is available from Superarray Bioscience Corporation, Frederick Md.

An in vitro test for PTEN missense mutations based on a phosphoinositide phosphatase assay is described in Cancer Res. 2000, Jun. 15; 60:, 3147-3151, which is incorporated herein by reference.

The test result of the performed assay are compared to a relevant threshold level. The relevant threshold level is determined for the tissue type tested and for the assay performed and reflects an average or lower value of PTEN expression. It will be appreciated that this threshold value is a balance between the likelihood of missing the opportunity to give appropriate therapy to a patient with a higher, but still reduced level of PTEN against the risk of treating a patient with a therapeutic that will not be effective resulting in a delay in administering alternative therapy. Thus, the specific threshold selected for any given cancer will depend on the variability of PTEN expression levels in non-cancerous “normal” tissues, the precision and accuracy of the assay employed, and the availability of viable alternative treatment modalities.

When the assay reveals an expression level of functional PTEN that is below the threshold level, a therapeutic composition comprising as an active agent a composition effective to inhibit the expression of hsp27 is administered to the patient. As noted above, inhibitors of hsp27 expression of various different types are known in the art. The specific route of administration, the dosage level and the treatment frequency will depend on the nature of the active agent employed. In general, the therapeutic agent may be administered by intravenous, intraperitoneal, subcutaneous, topical or oral routes, or direct local tumor injection. For example, antisense targeting hsp27 (such as gggacgcggc gctcggtcat, OGX-427, SEQ ID No. 1) may be administered at levels of injection at 200 mg, 400 mg, 600 mg, 800 mg or 1000 mg once a week as tolerated by the patient.

As discussed above, other inhibitors of hsp27 expression can also be employed, including evodiamine, which has the formula:

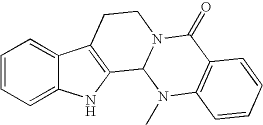

berberine derivative, magnolol-containing synthetic suppressors of protein belonging to hsp27 family, shikonin-containing synthetic suppressors of protein belonging to hsp27 family and aconitine-containing synthesis inhibitors of protein belonging to hsp27 family.

Having described the invention above, the following non-limiting examples are provided to further illustrate and demonstrate the invention. These experiments show that Hsp27 blockade selectively inhibits growth of PTEN deficient cancer cells and that Hsp27 chaperone is required for Akt stability and activity that, in turn, regulates phosphorylation and function of PEA-15. Hsp27 induces dual coordinated effects resulting in protection from Fas-induced apoptosis and promotion of cell proliferation through regulation of PEA-15 phosphorylation and function in an Akt dependent manner. Hsp27 overexpression resulted in activation of Akt and increased phosphorylation of its downstream target PEA-15 promoting enhanced ERK translocation to nucleus and increased Elk-1 activity which correlated with increased cyclin D1 and CDK2 expression with a concomitant decrease in p27Kip1 expression and increased cell proliferation. Furthermore, Hsp27 overexpression also led to increased association of PEA-15 with FADD and decreased sensitivity of cells to Fas-induced apoptosis . Conversely, Hsp27 knockdown led to reduced Akt activity and decreased phosphorylation of PEA-15 leading to reduced association of PEA-15 with FADD and increased sensitivity of cells to Fas induced apoptosis. Significantly, siRNA mediated Hsp27 knockdown in a panel of cell lines and in PTEN Tet-ON LNCaP cells that express PTEN in a doxycycline inducible manner demonstrated selective inhibition of growth of PTEN deficient cancer cells. These data identify a dual role of Hsp27 in regulating cell proliferation and Fas-induced apoptosis through regulation of PEA-15 and Akt and indicate that improved clinical responsiveness to Hsp27 targeted therapy can be achieved by stratification of patient populations based on expression of PTEN by cancer cells in accordance with the present invention.

### EXAMPLES

In the following examples, the materials and methods used were as follows:

### Cell Lines and Materials.

LNCaP, PC-3, DU145, 293T, Ku7, RT4, UMUC3, and MDA468 cells were purchased from American Type Culture Collection (ATCC, Rochville, Md., USA). PNT1b, LAPC4 and BPH-1 cells were a gift from Prof. N. Maitland (York, UK). LNCaP (used up to passage 50 in the present study), DU145, LAPC4, BPH-1, and PNT1b cells were routinely maintained in RPMI1640 (Life Technologies, Burlington, Ontario). RT4 cells were maintained in Macoy's media (Life Technologies, Burlington, Ontario). Other cells were maintained in DMEM (Life Technologies, Burlington, Ontario). Media were supplemented with 10% fetal bovine serum (FBS) and cultures were grown at 37° C. and 5% CO₂. GSK690693C kindly given by Dr. Rakesh Kumar was used as an AKT inhibitor in the present study. CH-11, anti-Fas antibody was purchased from Upstate. Cyclohexamide (CHX), doxycycline (Dox) and LY-294002 were purchased from Sigma. Hsp27 antibody, phospho-Hsp27 (Ser-82) antibody (StressGen), PEA-15 antibody (Santa Cruz Biotechnology), phospho-PEA15 (ser-116) antibody (Biospurce), Akt antibody, phospho-Akt (Ser-473) antibody, phospho-Foxol (Ser-256) antibody (Cell Signallig), FADD antibody (Upstate), p27, cyclin D1, CDK2 (Santa Cruz Biotechnology), Vinculin antibody (Sigma Chemical, Mo.) was purchased from each companies.

Lentiviral Infection of Hsp27 into LNCaP Cells

Two vectors, pHR′-CMV-Hsp27 and pHR′-CMV were used as an empty vector in the present study as previously described [Araujo, H., et al., J Biol Chem, 1993. 268(8): p. 5911-20.]: pHR-CMV-Hsp27 including the full-length cDNA for human Hsp27 was subcloned into the lentiviral vector pHR′-cytomegalovirus (CMV)-enhanced green fluorescent protein (EGFP) at the BamHI and XhoI sites. Infected LNCaP cells (LNCaPHsp27) were harvested for UV microscopy to verify green fluorescent protein expression, and Western blotting was used to verify Hsp27 expression.

Knockdown by siRNA Transfection

Twenty-four hours after culturing in 10 cm dishes at 7×105 cells per dish, Hsp27 siRNA (siHsp27) or scrambled siRNA (Scr) duplexes were transfected into cells. Briefly, the RNA duplex was diluted in Opti-MEN® serum-free medium and Oligofectamin (Invitrogen-Life Technologies). After 20 min, cells were transfected at 37? for 4-6 h and then were placed in standard medium. Forty-eight hours after transfection, cells were harvested and were analyzed in each experiment. To knockdown Akt, Akt1 siRNA (siAkt) duplexes (Cell signaling) were used. Twenty-four hours after transfection for 24 h, cells were harvested and then were analyzed as well. An siRNA duplex corresponding to the human Hsp27 site (sequence of one strand 5′-AAGUCUCAUCGGAUUUUGCAGC-3′, SEQ ID NO: 2; Dharmacon, Lafayette, Colo.) was used. A scrambled siRNA duplex (5′-CAGCGCUGACAACAGUUUCAU-3′ SEQ ID NO: 3) was used as a control.

### Western Blot Analysis

Proteins (20-40 μg/lane) extracted in RIPA buffer from cells, were electrophoresed in SDS-polyacrylamide gels (SDS/PAGE) and transferred to PDVF membranes (Milipore, Bedford, Mass.) by a wet transfer method. After blocking in TBST containing 5% nonfat milk powder at room temperature for an hour, membranes were incubated with the indicated antibodies at 4° C. overnight. After washing, membranes were then incubated for 30 min with 1:5000-diluted horseradish peroxidase-conjugate secondary antibodies (Santa Cruz Biotechnology, Calif.) at room temperature. Bands were detected by using an enhanced chemiluminescence Western blotting analysis system (Amersham Life Science, Arlington Heights, Ill.).

### Clonogenic Proliferation Assay

Cells were cultured at 0.5×10⁵ cells per well in 6 well-plates. Twenty-four hours after culture, cell growth was compared by a cell count method at 2 days intervals up to 7 days under standard medium. Each experiment was performed in 3 experiments.

### [³H]Thymidine Proliferation Assay

Cells were cultured at a concentration of 4×10⁴ cells per ml in 12 well-plates with standard medium for 24 h. On each day of the study at 24 h or 48 h after culture, 10 μl of 100 μCi/ml [³H]thymidine were added per well and cells were incubated for 3 h. The cells were detached from the plate with a trypsin-EDTA solution (0.05% trypsin and 0.53 mM EDTA; Life Technologies, Inc.). After centrifuging, cells were re-suspended in 100 μl ddH2O and were transferred to 96-well plates. The collected cells were counted on a Packard Top Count counter. Each experiment was performed in 6 experiments.

### Immunofluorescence

Cells were grown on glass coverslips in standard media for 24 h. Cells were fixed with cold 3% acetone in methanol for 10 min at −20° C. and permeabilized in 0.2% Triton in phosphate-buffered saline (PBS). Slides were incubated in blocking solution, 5% BSA in PBS for 1 h, and simultaneously treated overnight with primary antibodies, mouse monoclonal Hsp27 and rabbit polyclonal ERK antibodies. Secondary fluorescent antibodies, anti-mouse FITC and anti-rabbit Texas red conjugated were added for 1 h at room temperature with three 5-min washes (0.1% Triton in PBS). Cells examined for localization of red and green protein were mounted with fluorescent 4′,6-diamidino-2-phenylindole vectashield mounting medium (Vector Laboratories). Images were captured using a Zeiss Axioplan II fluorescence microscope (Zeiss) at ×100 magnification followed by analysis with imaging software (Northern Eclipse, Empix Imaging, Inc., Mississauga, Ontario, Canada). Analysis of focal co-localization was also done with Northern Eclipse and Adobe Photoshop CS software.

### Immunoprecipitation

Cell lysates were incubated with 5 μg Akt antibody or anti-IgG antibodies. After 12 h of incubation, 50 μL of protein A/G beads (Amersham Pharmacia Biosciences) were added into the reaction tubes and incubated for 2 h. The beads were washed three times using 1× PBS and resolved in 5× loading buffer (MBI, Fermentas Inc., Burlington, Canada). Hsp27 antibody was used and bands were detected as described above.

In vitro Akt Kinase Assay

Cells were harvested after culture and cell lysates were collected. Assessment of Akt activity was performed by the Akt kinase assay kit (Cell signaling), according to the manufacture's instructions. Briefly, 500 μg of proteins were incubated overnight with protein G-agarose beads bearing anti-Akt antibody on rotate at 4° C. to immunoprecipitate Akt. After washing, Akt-antibody-protein G-agarose complexes were added 1 μg of recombinant GSK-3A/B and 1 μl of magnesium/ATP mixture and were incubated for 30 min at 30°. After adding SDS sample buffer, samples were boiled for 5 min and were electrophoresed on 10% SDS-PAGE. The membranes were incubated with anti GSK-3A/B(ser21/9) and Akt antibodies. Vinculin expression of input was used as a control.

### Protein Stability

LNCaP cells treated with Scr or siHsp27 or LNCaPmock or LNCaPHsp27 cells were used. Media were changed 18-24 h later to RPMI +5% serum containing 10 μg/mL of cyclohexamide (CHX) incubated for the indicated time. Western blot was done by using Akt, Hsp27, and CAPDH antibodies.

### Elk-1 Transcription Reporter Assay

Cells were seeded onto 12-well plates at 105 cells per well. Cells grown in 6-well plates were transiently cotransfected in Opti-MEM® medium with lipofectin (Invitrogen), with 0.5 μg GAL-E1b-Luciferase reporter gene and a varying doses of pCMV-GAL4-Elk-1 kindly provided by Prof. Richard A. Maurer (Oregon, U.S.). Sixteen hours after, media was replaced with RPM11640 plus 10% FBS. Fluorescence was measured in a luminometer (MicroLumat Plus, EG & G Berhold) 48 h after transfection. Samples were normalized by cotransfection either pCMV-Renila or to protein concentration when Elk-1 effect was tested. Reporter assays were expressed in arbitrary light units and performed in 3 experiments.

CH-11-induced Apoptosis Assay

Cells were treated by 2.5 μg/ml CHX alone, 1000 ng/ml CH-11 alone or combined treatment with both drugs in 5% charcoal-stripped serum (CSS) media. Twenty hours later, cells were harvested and the percentage of subG1 populations were analyzed using flowcytometry. On the other hand, cells transfected with Scr 20 nM or siHsp27 20 nM were treated with 1000 ng/ml CH-11 in low-serum (0.5% FBS) media 48 h after each treatment. Apoptotic analysis was performed by using the percentage of subG1 populations after CH-11 administration by flowcytometry. Each assay was performed in 3 experiments.

### Statistical Analysis

All of the results were expressed as the mean±SD. Statistical analysis was done with a one-way ANOVA followed by Fisher's protected least significant difference test (StatView 512, Brain Power, Inc., Calabasas, Calif.). *, P<0.05 was considered significant.

### Example 1

### Hsp27 Regulates Akt Pathway in LNCaP Cells.

LNCaPHsp27 and LNCaP with empty vector (LNCaPmock) cells were assessed 24 h after culture. Cell lysates were collected 24 hours after culture and bands were detected with the indicated antibodies by Western blot assay. Hsp27 antibody, phospho-Hsp27 (Ser-82) antibody (StressGen), PEA15 antibody (Santa Cruz Biotechnology), phospho- PEA15 (ser-116) antibody (Biospurce), Akt antibody, phospho-Akt (Ser-473) antibody, phospho-Foxol (Ser-256) antibody (Cell Signaling) as a well-known downstream of AKT, were assessed both in LNCaP-M and Hsp27-LNCaP cells. Vinculin antibody (Sigma Chemical, Mo.) was used as a control of loading protein. Elevated Hsp27 and phospho-Hsp27 (Ser-82) protein levels in LNCaP cells stably expressing human Hsp27 cDNA (LNCaPHsp27) were observed. Akt and phospho-Akt (Ser-473), PEA15, phospho-PEA15 (ser116) and phospho-Foxol (Ser-256) known to be phosphorylated by Akt were up-regulated in LNCaPHsp27 cells. Hsp27 knockdown by siHsp27 led to down-regulation of Hsp27 in a dose-dependent manner. Next, effects of treatment by siAkt or GSK690693C, an Akt inhibitor, were assessed by Western blot assay in LNCaP cells. siAkt treatment down-regulated Akt, phospho-Akt PEA15, and phospho-PEA15, whereas GSK690693C treatment had a dose-dependent decrease of phospho-GSK-3B and phospho-PEA15 accompanied with up-regulation of phospho-Akt. These data indicates that Hsp27 regulates PEA15 phosphorylation via regulating Akt phosphorylation.

### Example 2

### Hsp27 Directly Interacts with Akt and Stabilizes Akt in LNCaP Cells

Hsp27 association with Akt was assessed by immunoprecipitation in LNCaP cells. Complete knockdown of Hsp27 abolished this interaction with Akt, whereas over-expression of Hsp27 increased the amount of Hsp27 immunoprecipitation with Akt. To estimate whether this interaction is concomitant with functional outcome, in vitro Akt kinase assay was examined. Knockdown of Hsp27 decreased Akt activity, while, increased Akt activity was seen in LNCaPHsp27 cells. The effect of Hsp27 knockdown on Akt stability was next evaluated by using CHX., which inhibits protein synthesis. Akt protein levels rapidly decrease after Hsp27 knockdown. In contrast, Hsp27 over-expression prolonged Akt half-life compared to LNCaP cells with empty vector. These data indicates that Hsp27 directly interacts with Akt and stabilizes Akt. This results provides a rational explanation for the importance of PTEN status to activity of Hsp27 inhibitors since PTEN inhibits formation of Akt, such that there is little or no Akt for the Hsp27 to interact with and stabilize.

### Example 3

### Hsp27 Levels Positively Correlate with ERK Translocation and Cell Proliferation Rates

Phospho-PEA15 is reported to stimulate translocation of ERK to the nucleus. Effect of Hsp27 levels on translocation of ERK in individual cells was estimated by immunofluorescent assay. Cell lysates from LNCaP-M cells and HSP27-LNCaP cells were collected 24 hours after culturing in standard media. LNCaP cells were treated with scramble (Scr) 20 nM or 20 nM hsp27 siRNA duplex (siHsp27) 20 nM as described above. Forty-eight hours after transfection, cells were harvested and cell lysates were collected.

Phospho-ERK antibody and ERK antibody (Santa Cruz Biotechnology) were used to detect bands. Eighteen hours after serum starvation, ERK staining in LNCaPmock cells was mainly localized to the cytoplasm as reflected in higher relative levels of ERP to phopho-ERK, but increased Hsp27 enhanced the translocation to the nucleus as reflected in increased relative amounts of phospo-ERK. Western blot assays also showed that Hsp27 over-expression increased ERK phosphorylation. Activity of Elk-1 as a representative transcription factor downstream of ERK was assessed by luciferase reporter assay. These assays demonstrated a dose-dependent and significant increase of Elk-1 activity in LNCaPHsp27 cells. In addition, shift of cell cycle-dependent molecules was investigated by western blot assay. Expression of Cyclin D1 and CDK2 increased and p27 expression decreased in LNCaPHsp27 cells, accounting for acceleration of cell cycle by Hsp27 over-expression.

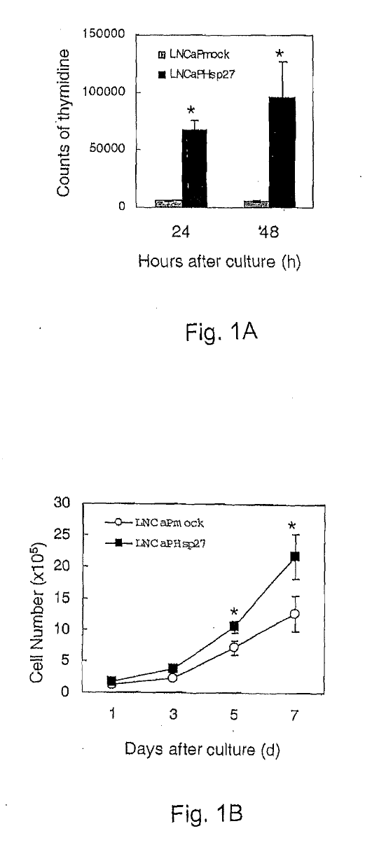

To identify whether this proliferation is accompanied with up-regulated DNA synthesis, [³H]Thymidine incorporation assays were used. [³H]Thymidine up-take in LNCaPHsp27 cells showed significant increase and reached to 20-fold counts up to 48 hours (FIG. 1A), accounting for accelerated DNA synthesis by Hsp27 over-expression. Moreover, clonogenic growth assay was performed by using a cell count method. LNCaPHsp27 cells showed significantly increased proliferation in time-dependent manner (FIG. 1B). These data demonstrates that increased Hsp27 functionally activates phosphorylation and nuclear translocation of ERK via PEA 15 and medicates increased cell proliferation in LNCaP cells.

### Example 4

### Hsp27 Regulates Induction of Fas-induced Apoptosis in LNCaP Cells

In addition to its effect on ERK translocation, Phospho-PEA15 displays anti-apoptotic effect through loss of FADD function by directly binding to FADD, a stimulator of Fas-mediated apoptosis. LNCaP cells are resistant to Fas stimuli. To study the involvement of Hsp27, apoptotic assays were performed using CHX, a protein synthesis inhibitor that also sensitizes cells to Fas-mediated apoptosis by degrading expression of c-FLIPL (cellular FLICE inhibitory protein long form).

Cytoprotective effect of Hsp27 to anti-Fas treatment was studied both in LNCaPHsp27 and LNCaPMock cells. LNCaPHsp27 and LNCaPMock cells were treated by 2.5 mg/ml CHX alone, 1000 ng/ml CH-11 alone and combined treatment with both drug in 5% CSS media. Apoptosis (% subG1 population) was assessed by flowcytometry 24 hours after treatment. B, Assessment of the effect of Hsp27 knockdown and CH-11 was performed 48 hours after transfection with scramble (Scr) 20 nM or Hsp27 siRNA duplex (siHsp27) 20 nM as described above. The effect of CH-11, 1000 ng/mL was compared with the presence or the absence of CH-11. Cell lysates from LNCaP-M and HSP27-LNCaP were collected 24 hours after culturing. Cell lysates from LNCaP cells treated with Scr 20 nM or siHsp27 20 nM were collected 48 hours after transfection. Proteins were incubated with PEA 15 antibody at 4° and bands were detected with FADD antibody (Upstate) by Western blot assay.

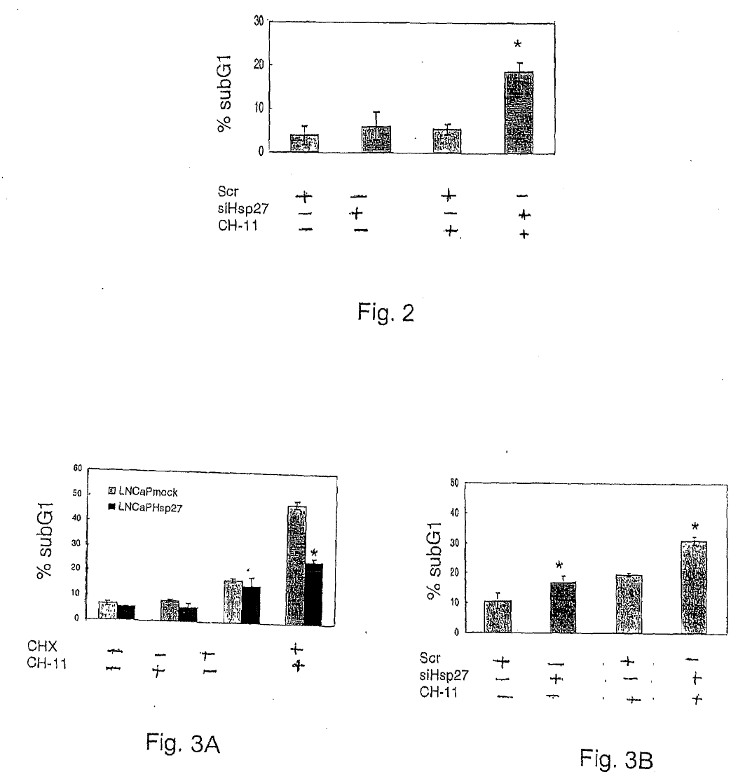

In LNCaPmock cells treated with CHX, apoptosis (% subG1 population) up to approximately 50% was induced by administration of CH-11. (FIGS. 2, 3A and B) In contrast, LNCaPHsp27 cells showed significantly less induced after the CH-11 treatment. Hsp27 knockdown by siHsp27 enhanced CH-11-induced apoptosis 48 hours after exposure in low-serum media in LNCaP cells. To estimate whether this protective effect to CH-11 treatment is involved in the association with FADD, immunoprecipitation with PEA15 was used. Hsp27 knockdown diminished the interaction with FADD, whereas increased Hsp27 strengthened this association. These data demonstrates that Hsp27 regulates the effect of Fas-induced apoptosis by modifying the interaction with FADD in LNCaP cells.

### Example 5

### Effects by Hsp27 Knockdown in PC-3 Cells

PC-3 prostate cancer cells were treated by Scr 20 nM or siHsp27 20 nM. Twenty-four hours after transfection, cells were fixed and were assessed by fluorescence microscopy after Hsp27 staining, ERK staining and DAPI nuclear staining. Hsp27 knockdown by siRNA decreased Akt and phospho- Akt (Ser-473), phospho-PEA15 (ser116) and phospho-Foxol (Ser-256) expression levels in dose-dependent manner in PC-3 cells. Next, changes of ERK localization after Hsp27 knockdown was assessed in PC-3 cells by immunofluorescence assay. Media was changed to a low serum (0.5%) condition after transfection. After 18 h, ERK nuclear accumulation clearly decreased after Hsp27 siRNA treatment, accounting for decreased phospho-PEA15 expression level. Hsp27 siRNA treatment sensitized PC-3 cells to Fas-mediated apoptosis.

### Example 6

### Correlation Between Hsp27 Knockdown Effect on Growth Rate and PTEN Status

Growth effects by Hsp27 knockdown were compared among 12 cell lines after transfection with scramble (Scr) 20 nM or Hsp27 siRNA (siHsp27) duplex 20 nM as described above. The cell growths were observed with 2 days intervals up to day7 after transfection. The date of transfection was considered as day 1. Protein extracts from cells 24 h after culture in standard media were assessed by Western blot assay using the indicated antibodies as described above. Cell number of control treated by Scr was considered as 100%.

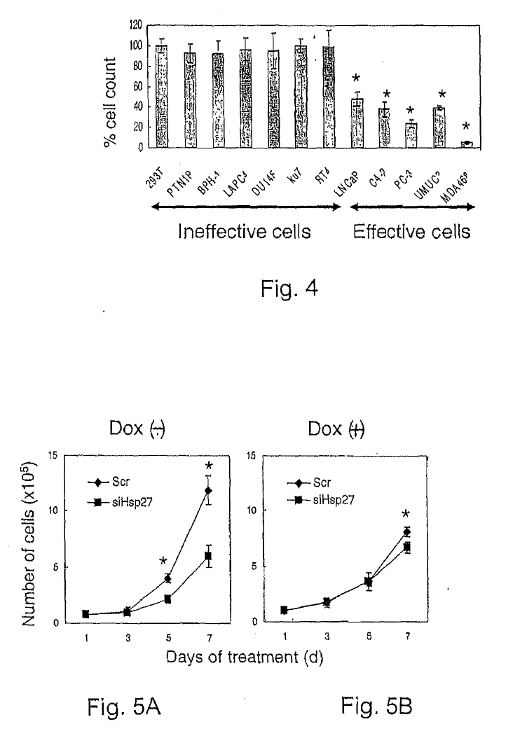

The relative growth rates for each cell type, represented as a percentage of cell count, 100 X (siHsp27/Scr), are shown in FIG. 4. As shown, the cells fell into two groups, those there the presence of siHsp27 had not effect, and those where it had a substantial inhibition on growth rate. The cell types in the first group (ineffective cells) are those with active PTEN. The cell types in the second group (effective cells) have inactive PTEN.

To further assess the relationship between PTEN status and growth inhibitory effect by siHsp27 treatment, LNCaP PTEN Tet-on inducible cells were used. Twenty-four hours after pre-culturing under the presence or absence of 1 μg/mL doxycycline (Dox), cells were treated with Scr 20 nM or siHsp27 20 nM and the cell growths were observed with 2 days intervals up to day 7. FIG. 5A shows the number of cells when doxycycline is absent and shows the ability of siHsp27 to reduce growth rate. In contrast, as shown in FIG. 5B, when doxycycline is present resulting in the induction of PTEN, the growth rates for the siHsp27 treated cells and the Scr treated cells are the same. Constitutive PTEN expression by Dox treatment was confirmed by Western blotting.

### Example 7

LY-294002 (2-(4-morpholinyl)-8-phenyl-4H-1-benzopyran-4-one) is a selective phosphatidylinositol 3-kinase (PI3K) inhibitor. Combination treatment with siHsp27 and LY-294002 treatment was assessed in PC-3 cells. LY-294002 treatment reduced PTEN expression and reduced the growth inhibitory effect by Hsp27 knockdown in dose-dependent manner. These data further demonstrate that growth inhibitory effect by Hsp27 knockdown is dependent on PTEN/Akt pathway via Hsp27 direct interaction with Akt.

All of the patents and publications referenced herein are incorporated herein by reference as though fully set forth herein.

1. A method for evaluation of a cancer, comprising the steps of:
(a) evaluating a sample of cancerous tissue to determine an expression of level of phosphatase and tensin homologue deleted from chromosome 10 (PTEN); and
(b) in the case where the expression level of functional PTEN is below a threshold level, identifying the cancer as susceptible to an active agent that inhibits the expression of heat shock protein 27 (hsp27).

2. The method of claim 1, wherein the step of evaluating the sample is performed using an antibody specific for PTEN.

3. The method of claim 1, wherein the step of evaluating the sample is performed using nucleotide sequencing techniques.

4. The method of claim 1, wherein the step of evaluating the sample is performed using polymerase chain reaction.

5. The method of claim 1, further comprising the step of evaluating a sample of the cancerous tissue for the expression of hsp27.

6. A method of for treating cancer in a patient diagnosed as suffering from cancer comprising the steps of:
(a) obtaining a sample of cancerous tissue from the patient;
(b) evaluating the sample of cancerous tissue to determine an expression of level of functional phosphatase and tensin homologue deleted from chromosome 10 (PTEN); and
(c) in the case where the expression level of functional PTEN is below a threshold level, administering to the patient a therapeutic composition comprising as an active agent a composition effective to inhibit the expression of heat shock protein 27 (hsp27).

7. The method of claim 6, wherein the step of evaluating the sample is performed using an antibody specific for PTEN.

8. The method of claim 6, wherein the step of evaluating the sample is performed using nucleotide sequencing techniques.

9. The method of claim 6, wherein the step of evaluating the sample is performed using polymerase chain reaction.

10. The method of claim 6, wherein the active agent is an oligonucleotide that interacts with cellular nucleic acids encoding hsp27 in a sequence specific manner.

11. The method of claim 10, wherein the oligonucleotide is an antisense oligonucleotide.

12. The method of claim 11, wherein the antisense oligonucleotide comprises SEQ ID NO. 1.

13. The method of claim 10, wherein the oligonucleotide is an siRNA oligonucleotide.

14. The method of claim 13, wherein the siRNA oligonucleotide comprises SEQ ID NO. 2.

15. The method of claim 6, wherein the active agent is a berberine derivative.

16. The method of claim 6, wherein the active agent is selected from the group consisting of magnolol-containing synthetic suppressors of protein belonging to hsp27 family, shikonin-containing synthetic suppressors of protein belonging to hsp27 family and aconitine-containing synthesis inhibitors of protein belonging to hsp27 family.

17. The method of claim 6, wherein the active agent comprises evodiamine.

18. The method of claim 6, further comprising the step of testing a sample from the patient for the expression of Hsp27 prior to performing the evaluation of step (b), wherein the sample tested for hsp27 expression can be the same as or different from the sample obtained in step (a).
